(19) United States
(12) Patent Application Publication
(10) Pub. No.: US 2017/0065511 A1
(43) Pub. Date: Mar. 9, 2017

(54) PROCESS FOR PRODUCING A CONDITIONING PRESHAVE OR AFTERSHAVE COMPOSITION

(71) Applicant: Henkel AG & Co. KGaA, Duesseldorf (DE)

(72) Inventors: Martina Kampmann, Korschenbroich (DE); Barbara Heide, Krefeld (DE)

(73) Assignee: Henkel AG & Co. KGaA, Duesseldorf (DE)

(21) Appl. No.: 15/353,996

(22) Filed: Nov. 17, 2016

Related U.S. Application Data
(63) Continuation of application No. PCT/EP2015/059895, filed on May 6, 2015.

(30) Foreign Application Priority Data
May 30, 2014 (DE) 10 2014 210 275.8

(51) Int. Cl. A61K 8/60; A61K 8/55; A61K 8/06; A61K 8/37; A61Q 19/00; A61Q 9/02

(52) U.S. Cl. CPC A61K 8/604; A61Q 19/002; A61Q 9/02; A61K 8/068; A61K 8/375; A61K 8/55

(57) ABSTRACT

The invention relates to a conditioning preshave/aftershave composition and related method. The composition includes a microemulsion including 15%-25% of at least one alkyl(oligo)glycoside, 4%-10% of at least one mono- or diester of glycerol with at least one C₁₀-C₂₄ fatty acid as coemulsifier, and 15%-25% at least one oil, and 40%-66% water. The composition can be mixed a water-containing cosmetic carrier comprising: at least one alkyl ether phosphate or alkenyl ether phosphate of the formula (I).

### FIELD OF THE INVENTION

The present invention generally relates to cosmetics, and more particularly relates to a process for producing a conditioning preshave or aftershave composition, in which a microemulsion is mixed with a cosmetic carrier comprising a special alk(en)yl ether phosphate emulsifier, water and oil.

### BACKGROUND OF THE INVENTION

The present invention further relates to a conditioning preshave or aftershave composition comprising a microemulsion, a special alk(en)yl ether phosphate emulsifier, water and oil, and to the non-therapeutic use of this conditioning preshave or aftershave composition for improving the skin sensation and/or for alleviating the sensation of tightness and of burning occurring as a result of shaving.

Cosmetic auxiliary shaving substances in the form of creams, aerosols, gels, soaps or foams have long been known and are continually further developed and/or adapted to the growing requirements of the consumer. They are usually applied to the skin just prior to shaving, in the form of so-called preshave compositions, or subsequent to shaving, in the form of so-called aftershave compositions.

Traditional shaving soaps often have the disadvantage that they cannot be optimally spread on the skin and irritate the skin. Gels and/or creams generally include special polymers and/or fats and are milder and easier to spread on the skin than traditional soaps. The additional nourishing agents, however, can result in excessive lubrication of the skin, so that the razor blade glides across the skin without completely removing the hair.

The application WO 95/05147 discloses, among other things, shaving foams that include a polyorganosiloxane mixture and leave behind a good feel of the skin before and after shaving. A major problem of shaving is the strong mechanical irritation of the skin and the unpleasant feel of the skin associated therewith. A particularly popular application form for shaving aids for shaving skin that is irritated, sensitive, tender, blemished and/or suffering from acne are shaving balms and shaving lotions that have a mid-range oil content. For the aforementioned skin types, it is particularly important to improve the feel of the skin, and in particular to alleviate the sensation of tightness and burning occurring as a result of shaving.

It was therefore the object of the present invention to provide shaving aids for shaving particularly sensitive skin (areas), since in the past the incorporation of larger amounts of emollients alone has not always been able to prevent skin irritations and/or small injuries of the skin that can be created by the razor blade during wet shaving.

The shaving aids were to impart sufficient pliability to the skin surface containing the hair so as to increase the ability of the razor blade to glide, without injuring the skin.

A further object was to produce a shaving aid that has a high skin nourishing effect and does not impart excessive lubrication to the skin during and/or after shaving.

It was a further object of the present invention to provide an uncomplicated process for producing a conditioning preshave or aftershave composition.

A further object of the present invention was to produce transparent preshave or aftershave compositions.

It was found that the combination of micro-emulsified oils with certain emulsifiers in shaving aids results in particularly nourishing products, which are easy to produce.

Furthermore, other desirable features and characteristics of the present invention will become apparent from the subsequent detailed description of the invention and the appended claims, taken in conjunction with this background of the invention.

### BRIEF SUMMARY OF THE INVENTION

A process for producing a conditioning preshave or aftershave composition, comprising the following steps: a) providing a microemulsion, comprising i) at least one alkyl(oligo)glycoside; ii) at least one mono- or diester of glycerol with at least one C₁₀ to C₂₄ fatty acid as the coemulsifier; iii) additionally at least one oil; and iv) water, wherein components (i), (ii), (iii) and (iv), based in each case on the weight of the microemulsion, are present in the amounts I, II, III and IV, with I=15% to 25% by weight, based on the weight of the microemulsion; II=4% to 10%/o by weight, based on the weight of the microemulsion; III=15% to 25% by weight, based on the weight of the microemulsion; IV=40% to 66% by weight, based on the weight of the microemulsion, and b) mixing the microemulsion with a water-containing cosmetic carrier comprising: i) as the emulsifier, at least one alkyl ether phosphate or alkenyl ether phosphate of formula (I),

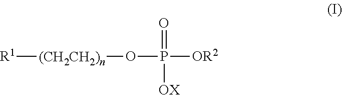

in which R¹ is an aliphatic hydrocarbyl radical having 8 to 30 carbon atoms, R² is hydrogen, a (CH₂CH₂O)ₙR¹ group or X, n is numbers from 1 to 10, and X is hydrogen, a (CH₂CH₂O)ₙR¹ group, an alkali metal or alkaline earth metal or NR³R⁴R⁵R⁶, where R³ to R⁶, independently of one another, are a C₁ to C₄ hydrocarbyl radical; and ii) at least one oil other than a) iii).

A conditioning preshave or aftershave composition, comprising: a) a microemulsion, comprising i) at least one alkyl(oligo)glycoside; ii) at least one mono- or diester of glycerol with at least one C₁₀-C₂₄ fatty acid as the coemulsifier; iii) at least one oil; and iv) water, wherein components (i), (ii), (iii) and (iv), based in each case on the weight of the microemulsion, are present in the amounts I, II, III and IV, with I=15% to 25% by weight, based on the weight of the microemulsion, II=4% to 10% by weight, based on the weight of the microemulsion, III=15% to 25% by weight, based on the weight of the microemulsion, IV=40% to 66% by weight, based on the weight of the microemulsion, furthermore at least one emulsifier of formula (I) in a total amount of 0.1 to 4% by weight, based on the total weight of the preshave or aftershave composition,

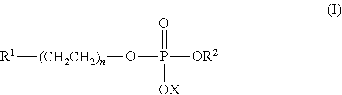

in which R¹ is an aliphatic hydrocarbyl radical having 8 to 30 carbon atoms, R² is hydrogen, a (CH₂CH₂O)ₙR¹ group or X, n is numbers from 1 to 10, and X is hydrogen, a (CH₂CH₂O)ₙR¹ group, an alkali metal or alkaline earth metal or NR³R⁴R⁵R⁶, where R³ to R⁶, independently of one another, are a C₁ to C₄ hydrocarbyl radical, and b) ii) is at least one oil other than a)iii), wherein the preshave or aftershave composition, in each case based on the total weight thereof, comprises in total 50 to 95% by weight water and in total 1 to 7% by weight oil(s).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the present invention is a process for producing a conditioning preshave or aftershave composition, comprising the following steps:
a) providing a microemulsion, comprising
   a) i) at least one alkyl(oligo)glycoside,
   a) ii) at least one mono- or diester of glycerol with at least one C₁₀-C₂₄ fatty acid as the coemulsifier, and additionally
   a) iii) at least one oil, and
   a) iv) water,
      wherein components (i), (ii), (iii) and (iv), based in each case on the weight of the microemulsion, are present in the amounts I, II, III and IV, with
      I=15% to 25% by weight, based on the weight of the microemulsion,
      II=4% to 10% by weight, based on the weight of the microemulsion,
      III=15% to 25% by weight, based on the weight of the microemulsion,
      IV=40% to 66% by weight, based on the weight of the microemulsion, and
b) mixing the microemulsion with a water-containing cosmetic carrier comprising:
   b) i) as the emulsifier, at least one alkyl ether phosphate or alkenyl ether phosphate of formula (I),

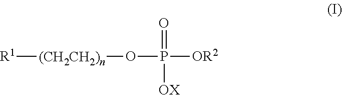

in which R¹ is an aliphatic hydrocarbyl radical having 8 to 30 carbon atoms, R² is hydrogen, a (CH₂CH₂O)ₙR¹ group or X, n is numbers from 1 to 10, and X is hydrogen, a (CH₂CH₂O)ₙR¹ group, an alkali metal or alkaline earth metal or NR³R⁴R⁵R⁶, where R³ to R⁶, independently of one another, are a C₁ to C₄ hydrocarbyl radical, and
b) ii) at least one oil other than a) iii).

Microemulsions a) that are suitable for use in the process according to the invention are preferably thermodynamically stable mixtures of oil, an emulsifier and a coemulsifier. Particularly preferred microemulsions have a volume average particulate size in the range of 10 nm to 150 nm, preferably of 20 nm to 100 nm, and particularly preferably of 30 to 80 nm.

A cosmetic carrier shall be understood to mean an aqueous or aqueous-alcoholic carrier.

In addition, the cosmetic water-containing carrier comprises, as an emulsifier, at least one alkyl ether phosphate or alkenyl ether phosphate of formula (I),

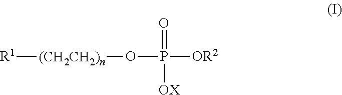

in which R¹ is an aliphatic hydrocarbyl radical having 8 to 30 carbon atoms, R² is hydrogen, a (CH₂CH₂O)ₙR¹ group or X, n is numbers from 1 to 10, and X is hydrogen, a (CH₂CH₂O)ₙR¹ group, an alkali metal or alkaline earth metal or NR³R⁴R⁵R⁶, where R³ to R⁶, independently of one another, are a C₁ to C₄ hydrocarbyl radical.

A preferred emulsifier of formula (I) is characterized in that the groups R¹ are at least one group selected from lauryl, myristyl, cetyl, stearyl or oleyl, and mixtures thereof; particularly preferred are emulsifiers of formula (I) in which the groups R¹ are the cetearyl mixture.

Further preferred emulsifiers of formula (I) are characterized in that the R² groups and X are a (CH₂CH₂O)ₙR¹ group, in which the groups R¹ are at least one group selected from lauryl, myristyl, cetyl, stearyl or oleyl, and mixtures thereof.

Further preferred emulsifiers of formula (I) are characterized in that n is numbers from 2 to 6, preferably 3 to 5, and particularly preferably 4.

Particularly preferred emulsifiers of formula (I) are characterized in that the groups R¹ are at least one group selected from lauryl, myristyl, cetyl, stearyl or oleyl, and mixtures thereof, and in particular the cetearyl mixture, and furthermore the groups R² and X are a (CH₂CH₂O)ₙR¹ group, wherein R¹ has the above meaning, and n is numbers from 2 to 6, preferably 3 to 5, and particularly preferably 4.

A particularly preferred process according to the invention is characterized in that the emulsifier of formula (I) is selected from trilaureth-4 phosphate and triceteareth-4 phosphate, and mixtures thereof. Exceptionally preferred is a process according to the invention in which the emulsifier of formula (I) is selected from triceteareth-4 phosphate.

The process according to the invention does not require a particular sequence when mixing components a) and b). In principle, it is possible to initially provide a mixture b) made of water, the at least one alkyl ether phosphate or alkenyl ether phosphate of formula (I) as the emulsifier and the at least one oil as a carrier, and to subsequently add the microemulsion a). Likewise, it is possible to add a mixture b) made of water, the at least one alkyl ether phosphate or alkenyl ether phosphate of formula (I) as the emulsifier and at least one oil to the microemulsion a).

In one preferred embodiment, initially a cosmetic carrier in the form of the above-described mixture b) made of water, the at least one alkyl ether phosphate or alkenyl ether phosphate of formula (I) as the emulsifier and at least one oil is provided. Thereafter, all optional components of the preshave or aftershave composition, such as perfume or preservatives, are incorporated into the carrier mixture. It may be preferred to produce the carrier mixture b) at a temperature in the range of 50 to 95° C. and to add the microemulsion a) at a temperature of 12 to 45° C., and in particular stir this in at a low shear rate of preferably 5 to 100 revolutions per minute.

Optionally, at the end, the viscosity of the preshave or aftershave composition is set to the desired range using a thickening agent.

Suitable alkyl ether phosphates or alkenyl ether phosphates according to formula (I) are preferably used in the process according to the invention in a total amount of 0.1 to 4% by weight, preferably 0.5 to 2% by weight, more preferably 0.8 to 1.5% by weight, each based on the total weight of the preshave or aftershave composition, wherein the quantity information is based on the total weight of the conditioning preshave or aftershave composition.

Microemulsions a) that are suitable for use in the process according to the invention comprise, in each case based on the total weight thereof,
(i) 15 to 25% by weight, more preferably 18 to 22% by weight of at least one alkyl(oligo)glycoside of the general formula RO-[G]ₓ, in which R is an alkyl and/or alkenyl group having 8 to 18 carbon atoms, preferably having 8 to 14 carbon atoms, G is a sugar residue having 5 or 6 carbon atoms, and in particular a glucose residue, and x is numbers from 1 to 5, and preferably numbers from 1 to 3;
(ii) 4 to 10% by weight, more preferably 6 to 9% by weight, and in particular 7 to 8% by weight of at least one monoester and/or diester of glycerol with a saturated or unsaturated, branched or unbranched C₁₀-C₂₄ fatty acid;
(iii) 15 to 25% by weight, more preferably 18 to 22% by weight of at least one oil; and
(iv) 40 to 66% by weight water.

Particularly suited alkyl(oligo)glycosides a(i) are derived from aldoses and/or ketoses having 5 or 6 carbon atoms, and preferably from glucose.

The group R particularly preferably is an alkyl group having 8 to 14, and in particular 10 to 12, carbon atoms, in particular n-octyl, n-decyl and lauryl.

The subscript x in the general formula RO-[G]ₓ indicates the degree of oligomerization (DP), which is to say the distribution of monoglycosides and oligoglycosides. The subscript x preferably has a value in the range of 1 to 5, and particularly preferably in the range of 1 to 3, wherein this may not be an integer, but a fractional number, which can be analytically ascertained.

Particularly preferred alkyl(oligo)glycosides have a degree of oligomerization of 1.2 to 1.5.

Particularly suited alkyl(oligo)glycosides are commercially available from various suppliers by the INCI names Decyl Glucoside, Lauryl Glucoside and Coco Glucoside.

Particularly suited esters a(ii) are monoesters of glycerol with linear fatty acids having alkyl chain lengths of 12 to 22 carbon atoms. Examples of particularly suited esters (ii) are glyceryl monolaurate, glyceryl monomyristate, glyceryl monopalmitate, glyceryl monostearate and/or glyceryl monooleate. Glyceryl monooleate is particularly suited.

Suitable oils a(iii) can be selected from mineral, natural and synthetic oil components and/or fats.

Preferred micro-emulsified oil components a(iii) are selected from dialkyl ethers. Particularly preferred dialkyl ethers are di-n-alkyl ethers having in total 12 to 36 carbon atoms, in particular 12 to 24 carbon atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether, and di-tert-butyl ether, di-iso-pentyl ether, di-3-ethyldecyl ether, tert-butyl-n-octyl ether, iso-pentyl-n-octyl ether and 2-methyl-pentyl-n-octyl ether. The di-n-octyl ether that is commercially available under the designation Cetiol® OE, for example, is particularly preferred.

Triglycerides and mixtures of triglycerides can be used as natural oils a(iii). Preferred natural oils are coconut oil, (sweet) almond oil, walnut oil, peach kernel oil, apricot kernel oil, avocado oil, tea tree oil, soy bean oil, sesame oil, sunflower oil, tsubaki oil, evening primrose oil, rice bran oil, palm kernel oil, mango kernel oil, lady's smock oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter and shea butter.

Mineral oils a(iii) that are used are in particular petroleum, paraffin and isoparaffin oils, and synthetic hydrocarbons. One example of a hydrocarbon that can be used, for example, is 1,3-di-(2-ethylhexyl)cyclohexane, which is available as a commercial product.

Silicone oils can be used as synthetic oils.

It is also possible to use fatty alcohols, and in particular branched and/or unsaturated fatty alcohols, as the oil component a(iii), for example octenol, dodecenol, decenol, octadienol, dodecadienol, decadienol, oleyl alcohol, eruca alcohol, ricinol alcohol, isostearyl alcohol, linoleyl alcohol, and linolenyl alcohol, and the Guerbet alcohols thereof.

Ester oils, for example, are further micro-emulsifiable oils a)iii). Ester oils shall be understood to mean the esters of C₆ to C₃₀ fatty acids with C₂ to C₃₀ fatty alcohols. The monoesters of fatty acids with alcohols having 2 to 24 carbon atoms are preferred. Examples of fatty acid components used in the esters are caproic acid, caprylic acid, 2-ethyl-hexanic acid, caprinic acid, lauric acid, isotridecanic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and the technical mixtures thereof.

Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, eleostearyl alcohol, arachyl alcohol, gadoleyl alcohol, behenyl alcohol, erucyl alcohol, and brassidyl alcohol, and the technical mixtures thereof. Particularly preferred are isopropyl myristate, isononanoic acid-C₁₆₋₁₈ alkyl ester, 2-ethylhexyl palmitate, stearic acid-2-ethylhexyl ester, cetyl oleate, glycerol tricaprylate, coconut fatty alcohol caprinate/caprylate, n-butyl stearate, oleyl erucate, isopropyl palmitate, oleyl oleate, lauric acid hexyl ester, di-n-butyl adipate, myristyl myristate, cetearyl isononanoate, and oleic acid decyl ester, and mixtures of these esters.

Further micro-emulsifiable oils a)iii) are, for example, dicarboxylic acid esters such as di-n-butyl adipate, di-(2-ethylhexyl) adipate, di-(2-ethylhexyl) succinate and diisotridecyl acelaate, and diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate.

Further micro-emulsifiable oils a)iii) are, for example, the symmetrical, asymmetrical or cyclic esters of carbonic acid with fatty alcohols, in particular glycerol carbonate or dicaprylyl carbonate.

Water is a further essential ingredient of the microemulsions a). The microemulsion a) comprises, based on the total weight thereof, 40 to 66% by weight, preferably 45 to 60% by weight, and in particular 50 to 55% by weight of water.

Particularly preferred microemulsions a) according to the invention comprise, in each case based on the weight thereof,
a) i) 15 to 25% by weight C₈-C₁₆ alkyl glucosides having a degree of oligomerization of 1.2 to 1.5;
a) ii) 4 to 10% by weight of a mixture of glyceryl monooleate and glyceryl dioleate;
a) iii) 15 to 25% by weight di-n-octyl ether; and
a) iv) 40 to 66% by weight water.

The microemulsion a) can preferably be produced (before the process according to the invention is carried out) by mixing the liquid oil phases (ii) and (iii) with the surfactant-containing, aqueous phase ((i) and (iv)) while stirring.

As an alternative, the microemulsion a) can also be used in the process according to the invention as a premanufactured commercial product. One example of a preferred microemulsion a) according to the invention that is commercially available is the microemulsion available under the designation “Plantasil Micro®” from Cognis.

The microemulsion a) is preferably used in the process according to the invention in an amount of 0.01 to 50% by weight, more preferably 0.1 to 30% by weight, particularly preferably 0.5 to 20% by weight, and in particular 1 to 15% by weight, wherein the quantities refer to the total weight of the conditioning preshave or aftershave composition.

At least one oil b)ii) other than the micro-emulsified oil a) iii) is present as a further mandatory component in the water-containing carrier mixture b) of the conditioning preshave or aftershave composition.

By selecting different oils, the application properties are optimized, in particular with respect to the ability to distribute the final product on the skin and the feel of the skin thus achieved. In principle, however, the oils a)iii) that are suitable for micro-emulsification, which may be selected from mineral, natural and synthetic oil components and/or fats, are also suitable as oils b)ii).

Preferred oil components b) ii) are selected from ester oils. Ester oils shall be understood to mean the esters of C₆ to C₃₀ fatty acids with C₂ to C₃₀ fatty alcohols. The monoesters of fatty acids with alcohols having 2 to 24 carbon atoms are preferred. Examples of fatty acid components used in the esters are caproic acid, caprylic acid, 2-ethyl-hexanic acid, caprinic acid, lauric acid, isotridecanic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid, and the technical mixtures thereof.

Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, eleostearyl alcohol, arachyl alcohol, gadoleyl alcohol, behenyl alcohol, erucyl alcohol, and brassidyl alcohol, and the technical mixtures thereof. Particularly preferred are isopropyl myristate, isononanoic acid-C₁₆₋₁₈ alkyl ester, 2-ethylhexyl palmitate, stearic acid-2-ethylhexyl ester, cetyl oleate, glycerol tricaprylate, coconut fatty alcohol caprinate/caprylate, n-butyl stearate, oleyl erucate, isopropyl palmitate, oleyl oleate, lauric acid hexyl ester, di-n-butyl adipate, myristyl myristate, cetearyl isononanoate, and oleic acid decyl ester, and mixtures of these esters. Particularly preferred ester oils are esters of C₁₂ to C₂₄ fatty acids with C₁₂ to C₂₄ fatty alcohols. These ester oils spread more slowly than the ester oils having fewer carbon atoms in the molecule and represent a good addition to the micro-emulsified oils having a lower molecular weight for the application properties of the end product.

Further suitable oils b)ii) are dicarboxylic acid esters such as di-n-butyl adipate, di-(2-ethylhexyl) adipate, di-(2-ethylhexyl) succinate and diisotridecyl acelaate, and diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate.

Further suitable oils b)ii) are the symmetrical, asymmetrical or cyclic esters of carbonic acid with fatty alcohols, in particular glycerol carbonate or dicaprylyl carbonate.

Further particularly suited oils b)ii) are selected from dialkyl ethers. Particularly preferred dialkyl ethers are di-n-alkyl ethers having in total 12 to 36 carbon atoms, in particular 12 to 24 carbon atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether, and di-tert-butyl ether, di-iso-pentyl ether, di-3-ethyldecyl ether, tert-butyl-n-octyl ether, iso-pentyl-n-octyl ether and 2-methyl-pentyl-n-octyl ether. The di-n-octyl ether that is commercially available under the designation Cetiol® OE, for example, is particularly preferred.

Triglycerides and mixtures of triglycerides can be used as natural oils b)ii). Preferred natural oils are coconut oil, (sweet) almond oil, walnut oil, peach kernel oil, apricot kernel oil, avocado oil, tea tree oil, soy bean oil, sesame oil, sunflower oil, tsubaki oil, evening primrose oil, rice bran oil, palm kernel oil, mango kernel oil, lady's smock oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter and/or shea butter.

Mineral oils b)ii) that are used are in particular petroleum, paraffin and isoparaffin oils, and synthetic hydrocarbons. One example of a hydrocarbon that can be used, for example, is 1,3-di-(2-ethylhexyl)cyclohexane, which is available as a commercial product.

Silicone oils can be used as synthetic oils b)ii).

It is also possible to use fatty alcohols, and in particular branched and/or unsaturated fatty alcohols, as the oil component b)ii), for example octenol, dodecenol, decenol, octadienol, dodecadienol, decadienol, oleyl alcohol, eruca alcohol, ricinol alcohol, isostearyl alcohol, linoleyl alcohol, and linolenyl alcohol, and the Guerbet alcohols thereof.

In a particularly preferred embodiment, the carrier mixture b) can also comprise the oil a)iii) in not micro-emulsified form, in addition to the at least one oil b)ii), which is also different from the at least one micro-emulsified oil a)iii).

In a further particularly preferred embodiment, the preshave or aftershave composition produced according to the invention, or the preshave or aftershave composition according to the invention, in total includes, in each case based on the total weight thereof, 1 to 7% by weight oils, preferably 2 to 60% by weight oils, and particularly preferably 3 to 5% by weight oils, including all micro-emulsified and not micro-emulsified oils.

In a further particularly preferred embodiment, the preshave or aftershave composition produced according to the invention, or the preshave or aftershave composition according to the invention, in total includes, in each case based on the total weight thereof, 0.05 to 5% by weight, preferably 0.1 to 3% by weight, and particularly preferably 0.2 to 1.5% by weight of one or more oils in micro-emulsified form.

In a further particularly preferred embodiment, the preshave or aftershave composition produced according to the invention, or the preshave or aftershave composition according to the invention, in total includes, in each case based on the total weight thereof, 0.95 to 6.95% by weight, preferably 2 to 5% by weight, and particularly preferably 3 to 4% by weight of one or more oils in not micro-emulsified form.

In a further particularly preferred embodiment, the preshave or aftershave composition produced according to the invention, or the preshave or aftershave composition according to the invention, in total includes, in each case based on the total weight thereof, 1 to 7% by weight oils, preferably 2 to 6% by weight oils, and particularly preferably 3 to 5% by weight oils, of which 0.05 to 5% by weight, preferably 0.1 to 3% by weight, and particularly preferably 0.2 to 1.5% by weight is one or more oils in micro-emulsified form, and 0.95 to 6.95% by weight, preferably 2 to 5% by weight, and particularly preferably 3 to 4% by weight of one or more oils in not micro-emulsified form.

In a particularly preferred embodiment, the production process according to the invention results in conditioning preshave or aftershave compositions that are transparent.

“Transparent” shall be understood to mean that the conditioning preshave or aftershave compositions have a nephelometric turbidity unit (NTU) value at 20° C. of no more than 100, preferably of no more than 50, and in particular of no more than 30.

So as to preserve the stability and the special nourishing properties of the microemulsion a) after mixing with the carrier mixture b), it may be preferred to limit the surfactant content of the carrier mixture b), apart from the at least one alkyl ether phosphate or alkenyl ether phosphate of formula (I) as the emulsifier b) i), and limit the addition of further surfactants in terms of the amount and type of the surfactants.

In a preferred embodiment, the microemulsion a) does not comprise any further surfactants outside the 15 to 25% by weight of alkyl(oligo)glycoside(s).

In a further preferred embodiment, the conditioning preshave or aftershave composition, in addition to the at least one alkyl ether phosphate or alkenyl ether phosphate of formula (I) as the emulsifier b) i) and the surfactants from the microemulsion a), comprises further surfactants in a total amount of 0 to 1.5% by weight, preferably 0.01 to 1% by weight, and more preferably 0.1 to 0.3% by weight, in each case based on the total weight of the preshave or aftershave composition.

The optional anionic surfactants include:
linear and branched fatty acids having 8 to 30 carbon atoms (soaps);
ether carboxylic acids of formula R—O—(CH₂—CH₂O)ₓ—CH₂—COOH, in which R is a linear or branched, saturated or unsaturated alkyl group having 8 to 30 carbon atoms, and x=0 or 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
sulfosuccinic acid monoalkyl and/or dialkyl esters having 8 to 24 carbon atoms in the alkyl group, and sulfosuccinic acid monoalkyl polyoxyethyl esters having 8 to 24 carbon atoms in the alkyl group and 1 to 6 oxyethyl groups;
alpha-olefin sulfonates having 8 to 24 carbon atoms;
alkyl sulfate and/or alkyl polyglycol ether sulfate salts of formula R—O—(CH₂—CH₂O)ₓ—OSO₃₋X⁺, in which R is a preferably linear or branched, saturated or unsaturated alkyl group having 8 to 30 carbon atoms, x=0 or 1 to 12, and X is an alkali or ammonium ion;
sulfonates of unsaturated fatty acids having 8 to 24 carbon atoms and 1 to 6 double bonds;
esters of tartaric acid and citric acid with alcohols that represent addition products of approximately 2 to 15 molecules ethylene oxide and/or propylene oxide to fatty alcohols having 8 to 22 carbon atoms.

Optional amphoteric/zwitterionic surfactants can be selected from compounds of the following formulas (i) to (v), in which the group R is a straight-chain or branched, saturated or monounsaturated or polyunsaturated alkyl or alkenyl group having 8 to 24 carbon atoms.

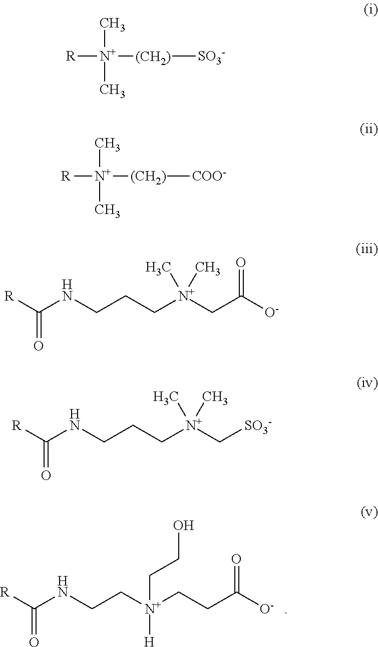

Particularly suited optional amphoteric/zwitterionic surfactants are alkylamidoalkyl betaines and/or alkylampho(di)acetates of the above-mentioned formulas (i) to (v), and in particular the surfactants known by the INCI names Cocamidopropyl Betaine and Disodium Cocoamphodiacetate.

Optional non-ionic surfactants/emulsifiers are, for example,
C₈ to C₃₀ fatty acid monoesters and diesters of addition products of 1 to 30 moles ethylene oxide to glycerol, for example that known by the INCI name PEG-7 Glyceryl Cocoate;
amine oxides;
addition products of 2 to 50 moles ethylene oxide and/or 0 to 5 moles propylene oxide to linear and branched fatty alcohols having 8 to 30 carbon atoms, to fatty acids having 8 to 30 carbon atoms, and to alkyl phenols having 8 to 15 carbon atoms in the alkyl group;
sorbitan fatty acid esters and addition products of ethylene oxide to sorbitan fatty acid esters, such as polysorbates;
sugar fatty acid esters and addition products of ethylene oxide to sugar fatty acid esters; and/or
addition products of ethylene oxide to fatty acid alkanolamides and fatty amines.

The nourishing action of the conditioning preshave or aftershave compositions produced by the process according to the invention is achieved by homogeneously distributing and depositing the lipid component(s) from the microemulsion on the skin. Surprisingly, this noticeably alleviates the burning and the irritated sensation of the skin irritated by the shaving process. The use as a preshave composition additionally facilitates the razor blade gliding across the skin, without making the skin too slippery and thereby impairing the result of the shaving process.

The mandatory components of the carrier mixture b), which is to say the at least one alkyl ether phosphate or alkenyl ether phosphate of formula (I) as the emulsifier b) i) and the at least one oil, other than the micro-emulsified oil a) iii), contribute to the skin-soothing and nourishing effect.

The conditioning preshave or aftershave composition according to the invention may include a number of other optional active ingredients, which can bring about advantageous properties on the hair and do not impede the process according to the invention. The preferred optional active ingredients include, for example: thickening agents.

Particularly preferred thickening agents for the conditioning preshave or aftershave composition produced according to the invention, or the conditioning preshave or aftershave composition according to the invention, are selected from at least one homopolymer or copolymer of acrylic acid, 2-methyl-2-[(1-oxo-2-propenyl)amino]-1-propanesulfonic acid (AMPS), acrylic acid amides or acrylic acid esters, and mixtures of the aforementioned monomers. Particularly preferred production processes and compositions are characterized in that the at least one homopolymer or copolymer of acrylic acid, 2-methyl-2-[(1-oxo-2-propenyl)amino]-1-propanesulfonic acid, acrylic acid amides or acrylic acid esters b) ii) is selected from polyacrylic acid, the salts of polyacrylic acid, homopolymers of 2-methyl-2-[(1-oxo-2-propenyl)amino]-1-propanesulfonic acid, copolymers of 2-methyl-2-[(1-oxo-2-propenyl)amino]-1-propanesulfonic acid and acrylamide, copolymers of 2-methyl-2-[(1-oxo-2-propenyl)amino]-1-propanesulfonic acid and acrylic acid, copolymers of 2-methyl-2-[(1-oxo-2-propenyl)amino]-1-propanesulfonic acid and 2-hydroxyethyl acrylate, copolymers of 2-methyl-2-[(1-oxo-2-propenyl)amino]-1-propanesulfonic acid and polyvinylpyrrolidone, and mixtures thereof. Further particularly preferred production processes and compositions are characterized in that the conditioning preshave or aftershave composition produced according to the invention, or the conditioning preshave or aftershave composition according to the invention, comprises at least one thickening agent, selected from at least one homopolymer or copolymer of acrylic acid, 2-methyl-2-[(1-oxo-2-propenyl)amino]-1-propanesulfonic acid, acrylic acid amides or acrylic acid esters, and from mixtures of the aforementioned monomers, in a total amount of 0.01 to 1.5% by weight, preferably 0.1 to 1.1% by weight, and particularly preferably 0.3 to 0.8% by weight, each based on the weight of the conditioning preshave or aftershave composition. Preferred conditioning preshave or aftershave compositions have a viscosity in the range of 2000 to 30000 mPas, and preferably of 5000 to 15000 mPas, measured at 20° C. by way of a rotational viscometer.
vitamins, vitamin derivatives and/or vitamin precursors, which may preferably be used in the process according to the invention in an amount of 0.001 to 3% by weight, more preferably 0.005 to 2% by weight, and in particular 0.01 to 1% by weight, wherein the quantity information in each case refers to the total weight of the conditioning preshave or aftershave composition.

Suitable vitamins shall preferably be understood to mean the following vitamins, provitamins, and vitamin precursors, and the derivatives thereof:

Vitamin A: The group of substances referred to as vitamin A includes retinol (vitamin A₁) and 3,4-didehydroretinol (vitamin A₂). β-carotene is the provitamin of retinol. Possible vitamin A components are, for example, vitamin A acid and the esters thereof, vitamin A aldehyde, and vitamin A alcohol and the esters thereof, such as palmitate and acetate. Vitamin B: The vitamin B group or vitamin B complex includes, among other things.
Vitamin B₁ (thiamine)
Vitamin B₂ (riboflavin)
Vitamin B₃. The compounds nicotinic acid and nicotinic acid amide (niacinamide) are often covered by this designation.
Vitamin B₅ (pantothenic acid and panthenol). Within this group, panthenol is preferably used. Derivatives of panthenol that may be used are in particular the esters and ethers of panthenol and cationically derivatized panthenols. Individual representatives are, for example, panthenol triacetate, panthenol monoethyl ether and the monoacetate thereof, and cationic panthenol derivatives.
Vitamin B₆ (pyridoxine, pyridoxamine and pyridoxal).

Vitamin C (ascorbic acid): Use in the form of the palmitic acid ester, glucosides or phosphates can be preferred. Use in combination with tocopherols can likewise be preferred.

Vitamin E (tocopherols, in particular α-tocopherol).

Vitamin F: The term “vitamin F” is usually understood to mean essential fatty acids, in particular linoleic acid, linolenic acid and arachidonic acid.

Vitamin H: Vitamin H refers to the compound (3aS,4S,6aR)-2-oxohexahydrothienol[3,4-d]-imidazole-4-valeric acid, which has since become established by the trivial name biotin.

Other active ingredients, auxiliary agents and additives that may be used in the process according to the invention are, for example, plant extracts, humectants, perfumes, UV filters, dyes for coloring the composition, substances for setting the pH value, for example alpha- and beta-hydroxycarboxylic acids such as citric acid, lactic acid, malic acid, glycolic acid, active ingredients such as bisabolol or allantoin, complexing agents such as EDTA, NTA, 3-alaninediacetic acid, and phosphonic acids, antioxidants, and preservatives, such as sodium benzoate or salicylic acid.

The process according to the invention is preferably suited for the production of conditioning preshave or aftershave compositions that have a pH value in the range of 4.0 to 7.5, preferably of 4.5 to 6.5, and in particular of 4.5 to 5.0.

The process according to the invention has the advantage that it is particularly easy to carry out and requires low energy expenditure. The nourishing components a) and b) can be mixed with each other in any arbitrary sequence, and further auxiliary agents and active ingredients can be incorporated into the cosmetic carrier without making the process notably more complicated.

Furthermore, the process according to the invention can be used to produce conditioning preshave or aftershave compositions that are transparent and stable.

The skin treatment using the conditioning preshave or aftershave composition according to the invention, which is preferably accessible via the process according to the invention or a preferred process according to the invention, brings about an improvement in the skin sensation and/or an alleviation of the sensation of tightness and burning that occurs as a result of shaving.

A second subject matter of the invention is a conditioning preshave or aftershave composition, comprising:
a) a microemulsion, comprising
   a) i) at least one alkyl(oligo)glycoside,
   a) ii) at least one mono- or diester of glycerol with at least one C₁₀-C₂₄ fatty acid as the coemulsifier,
   a) iii) at least one oil, and
   a) iv) water,
wherein components (i), (ii), (iii) and (iv), based in each case on the weight of the microemulsion, are present in the amounts I, II, III and IV, with
I=15% to 25% by weight, based on the weight of the microemulsion,
II=40% to 10% by weight, based on the weight of the microemulsion,
III=15% to 25% by weight, based on the weight of the microemulsion,
IV=40% to 66% by weight, based on the weight of the microemulsion,
furthermore
at least one emulsifier of formula (I) in a total amount of 0.1 to 4% by weight, based on the total weight of the preshave or aftershave composition,

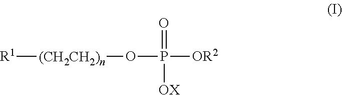

in which R¹ is an aliphatic hydrocarbyl radical having 8 to 30 carbon atoms, R² is hydrogen, a (CH₂CH₂O)ₙR¹ group or X, n is numbers from 1 to 10, and X is hydrogen, a (CH₂CH₂O)ₙR¹ group, an alkali metal or alkaline earth metal or NR³R⁴R⁵R⁶, where R³ to R⁶, independently of one another, are a C₁ to C₄ hydrocarbyl radical, and b) ii) is at least one oil other than a)iii),
wherein the preshave or aftershave composition, in each case based on the total weight thereof, comprises in total 50 to 95% by weight water and in total 1 to 7% by weight oil(s).

A third subject matter of the invention is the use of the above-described cosmetic preshave or aftershave composition to improve the skin sensation and/or to alleviate the sensation of tightness and burning that occurs as a result of shaving.

What was said above with respect to the process according to the invention applies, mutatis mutandis, with respect to further preferred embodiments of the composition according to the invention and the use according to the invention.

### Examples

### Conditioning Aftershave (all Quantity Information is in % by Weight, Based on the Aftershave Composition)

| | |
|---|---|
| Triceteareth-4 phosphate | 1.0 |
| Propyl paraben | 0.2 |
| Di-n-octyl ether (not micro-emulsified) | 1 |
| Erucic acid oleyl ester (oleyl erucate, not micro- | 2 |
| emulsified) | |
| 1,6-hexanediol | 6 |
| Methyl paraben | 0.2 |
| Glycerol 99.5% by weight | 3 |
| Water (carrier mixture b) | 80.94 |
| Lactic acid 80% by weight DAB | 0.06 |
| Perfume | 0.6 |
| Microemulsion, therein | 3 |
| Water (ex microemulsion) | 1.6 |
| Di-n-octyl ether (micro-emulsified) | 0.6 |
| Glyceryl monooleate/dioleate (ex microemulsion) | 0.2 |
| C₈₋₁₆ alkyl polyglucoside (ex microemulsion, | 0.6 |
| INCI: Decyl Glucoside) | |
| Sepigel 305 (from Seppic) | 2.0 |
| Water (ex Sepigel 305) | 0.7 |
| AMPS/polyacrylamide copolymer (ex Sepigel 305) | 0.8 |
| Laureth-7 (ex Sepigel 305) | 0.1 |
| C₁₃₋₁₄ isoparaffin (ex Sepigel 305) | 0.4 |

Production:

Using simple mixing at a temperature in the range of 15 to 35° C., a microemulsion was produced from the following components listed in the table: water, di-n-octyl ether, glyceryl monooleate/dioleate, and C₈₋₁₆ alkyl polyglucoside (INCI: Decyl Glucoside). Separately therefrom triceteareth-4 phosphate, propyl paraben, di-n-octyl ether (not micro-emulsified), and oleyl erucate (not micro-emulsified) were melted together, mixed, and heated to 90° C. (->oil phase).

The water (carrier mixture b) was mixed with glycerol, 1,6-hexanediol and methyl paraben and heated to 90° C. (->water phase).

The hot oil phase and the hot water phase were mixed with each other and homogenized to form an emulsion, which represents the carrier mixture b.

This carrier mixture b was cooled to 40° C. while stirring. Thereafter, the microemulsion, the perfume, and the lactic acid were added.

Finally, the thickening agent preparation (Sepigel 305) was added.

The conditioning aftershave composition thus produced had a pH value of approximately 4.6, measured at 20° C.

The viscosity was approximately 6000 mPas, measured at 20° C. by way of rotational viscometer.

The conditioning aftershave composition was applied to the shaved facial skin directly after shaving. All test subjects felt a sensation of tightness and burning of the skin after shaving, but prior to applying the aftershave composition. Applying the conditioning aftershave composition according to the invention provided immediate relief of the sensation of tightness and burning. Furthermore, the test subjects stated that they felt an improved, soft, not tight skin sensation 30 minutes after applying the aftershave composition.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A process for producing a conditioning preshave or aftershave composition, comprising the following steps:
a) providing a microemulsion, comprising
a) i) at least one alkyl(oligo)glycoside,
a) ii) at least one mono- or diester of glycerol with at least one C₁₀ to C₂₄ fatty acid as the coemulsifier, additionally
a) iii) at least one oil, and
a) iv) water,
wherein components (i), (ii), (iii) and (iv), based in each case on the weight of the microemulsion, are present in the amounts I, II, III and IV, with
I=15% to 25% by weight, based on the weight of the microemulsion,
II=4% to 10% by weight, based on the weight of the microemulsion,
III=15% to 25% by weight, based on the weight of the microemulsion,
IV=40% to 660% by weight, based on the weight of the microemulsion,
and
b) mixing the microemulsion with a water-containing cosmetic carrier comprising:
b) i) as the emulsifier, at least one alkyl ether phosphate or alkenyl ether phosphate of formula (I), 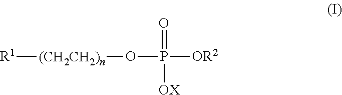
in which R¹ is an aliphatic hydrocarbyl radical having 8 to 30 carbon atoms, R² is hydrogen, a (CH₂CH₂O)ₙR¹ group or X, n is numbers from 1 to 10, and X is hydrogen, a (CH₂CH₂O)ₙR¹ group, an alkali metal or alkaline earth metal or NR³R⁴R⁵R⁶, where R³ to R⁶, independently of one another, are a C₁ to C₄ hydrocarbyl radical,
b) ii) at least one oil other than a) iii).

2. The process according to claim 1, wherein for formula (I)
the groups R¹ include at least one group selected from lauryl, myristyl, cetyl, stearyl or oleyl, and mixtures thereof,
the groups R² and X are a (CH₂CH₂O)ₙR¹ group, and
n is numbers from 2 to 6.

3. The process according to claim 1, wherein the emulsifier of formula (I) is selected from the group consisting of trilaureth-4 phosphate, triceteareth-4 phosphate, and mixtures thereof.

4. The process according to claim 1, wherein the conditioning preshave or aftershave composition comprises the at least one emulsifier of formula (I) in a total amount of 0.1 to 4% by weight based on the total weight of the preshave or aftershave composition.

5. The process according to claim 1, wherein the microemulsion a) comprises
(i) in total 15 to 25% by weight of at least one alkyl(oligo)glycoside of the general formula RO-[G]x, in which R is an alkyl and/or alkenyl group having 4 to 22 carbon atoms, G is a sugar residue having 5 or 6 carbon atoms, and x is numbers from 1 to 10;
(ii) in total 4 to 10% by weight of at least one monoester and/or diester of glycerol with a saturated or unsaturated, branched or unbranched C₁₀-C₂₄ fatty acid;
(iii) in total 15 to 25% by weight of at least one oil other than a)ii); and
(iv) 40 to 66% by weight water,
wherein the quantity information is based on the weight of the microemulsion a).

6. The process according to claim 1, wherein the conditioning preshave or aftershave composition, based on the total weight thereof, comprises 0.5 to 50% by weight of the microemulsion a).

7. A conditioning preshave or aftershave composition, comprising:
a) a microemulsion, comprising
a) i) at least one alkyl(oligo)glycoside,
a) ii) at least one mono- or diester of glycerol with at least one C₁₀-C₂₄ fatty acid as the coemulsifier,
a) iii) at least one oil, and
a) iv) water,
wherein components (i), (ii), (iii) and (iv), based in each case on the weight of the microemulsion, are present in the amounts I, II, III and IV, with
I=15% to 25% by weight, based on the weight of the microemulsion,
II=4% to 10% by weight, based on the weight of the microemulsion,
III=15% to 25% by weight, based on the weight of the microemulsion,
IV=40% to 66% by weight, based on the weight of the microemulsion,
furthermore
at least one emulsifier of formula (I) in a total amount of 0.1 to 4% by weight, based on the total weight of the preshave or aftershave composition, 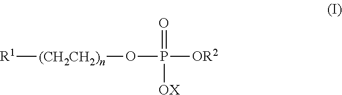
in which R¹ is an aliphatic hydrocarbyl radical having 8 to 30 carbon atoms, R² is hydrogen, a (CH₂CH₂O)ₙR¹ group or X, n is numbers from 1 to 10, and X is hydrogen, a (CH₂CH₂O)ₙR¹ group, an alkali metal or alkaline earth metal or NR³R⁴R⁵R⁶, where R³ to R⁶, independently of one another, are a C₁ to C₄ hydrocarbyl radical, and b) ii) is at least one oil other than a)iii),
wherein the preshave or aftershave composition, in each case based on the total weight thereof, comprises in total 50 to 95% by weight water and in total 1 to 7% by weight oil(s).

8. The conditioning preshave or aftershave composition according to claim 7, wherein the microemulsion a), in each case based on the weight thereof, comprises
a) i) 15 to 25% weight C₈-C₁₆ alkyl glucosides having a degree of oligomerization of 1.2 to 1.5;
a) ii) 4 to 10% by weight of a mixture of glyceryl monooleate and glyceryl dioleate;
a) iii) 15 to 25% by weight di-n-octyl ether; and
a) iv) 40 to 66% by weight water.

9. The conditioning preshave or aftershave composition according to claim 7, wherein the microemulsion a), except for the 15 to 25% by weight of alkyl(oligo)glycoside(s), does not comprise any further surfactants.

10. The conditioning preshave or aftershave composition according to claim 7, wherein the conditioning preshave or aftershave composition, in addition to the at least one alkyl ether phosphate or alkenyl ether phosphate of formula (I) as the emulsifier b) i) and the surfactants from the microemulsion a), comprises further surfactants in a total amount of 0 to 1.5% by weight, based on the total weight of the preshave or aftershave composition.

11. The conditioning preshave or aftershave composition according to claim 7, wherein the preshave or aftershave composition comprises in total, in each case based on the total weight thereof, 1 to 70% by weight oils including all micro-emulsified oils.

12. The conditioning preshave or aftershave composition according to claim 7, wherein the preshave or aftershave composition comprises in total, in each case based on the total weight thereof, 2 to 60% by weight oils including all micro-emulsified oils.

13. The conditioning preshave or aftershave composition according to claim 7, wherein the preshave or aftershave composition comprises in total, in each case based on the total weight thereof, 3 to 5% by weight oils including all micro-emulsified oils.

14. The conditioning preshave or aftershave composition according to claim 7, characterized in that the preshave or aftershave composition comprises in total, in each case based on the total weight thereof, 0.05 to 5% by weight of one or more oils in micro-emulsified form.

15. The conditioning preshave or aftershave composition according to claim 7, characterized in that the preshave or aftershave composition comprises in total, in each case based on the total weight thereof, 0.1 to 3% by weight of one or more oils in micro-emulsified form.

16. The conditioning preshave or aftershave composition according to claim 7, characterized in that the preshave or aftershave composition comprises in total, in each case based on the total weight thereof, 0.2 to 1.5% by weight of one or more oils in micro-emulsified form.

17. The conditioning preshave or aftershave composition according to claim 7, characterized in that the preshave or aftershave composition comprises in total, in each case based on the total weight thereof, 0.95 to 6.95% by weight of one or more oils not in micro-emulsified form.

18. The conditioning preshave or aftershave composition according to claim 7, characterized in that the preshave or aftershave composition comprises in total, in each case based on the total weight thereof, 2 to 5% by weight of one or more oils not in micro-emulsified form.

19. The conditioning preshave or aftershave composition according to claim 7, characterized in that the preshave or aftershave composition comprises in total, in each case based on the total weight thereof, 3 to 4% by weight of one or more oils not in micro-emulsified form.

20. A non-therapeutic use of a conditioning preshave or aftershave composition, comprising: applying to a skin surface the composition of claim 7 prior to or after shaving.
